(19) United States
(12) Patent Application Publication
(10) Pub. No.: US 2012/0053405 A1
(43) Pub. Date: Mar. 1, 2012

(54) FORCEPS TOOL FOR ENDOSCOPE

(75) Inventor: Noriyuki SUGITA, Saitama (JP)

(73) Assignee: HOYA CORPORATION, Tokyo (JP)

(21) Appl. No.: 13/204,944

(22) Filed: Aug. 8, 2011

(30) Foreign Application Priority Data
Aug. 24, 2010 (JP) 2010-186991

(51) Int. Cl. A61B 17/94

(52) U.S. Cl. 600/106

(57) ABSTRACT

A forceps tool for an endoscope is provided with a tube member, a support member secured to a distal end of the tube member, a support pin provided to the support member, a pair of forceps rotatably supported by the support pin, the pair of forceps being rotated about the support pin, a cam groove being formed on each of the pair of forceps, a movable member configured to axially move inside the tube in an axial direction thereof, an insertion hole formed on the movable member, a cam pin inserted through the insertion hole, both ends of the cam pin engaging with the cam grooves, respectively. The pair of forceps rotates about the support pin in accordance with engagement of the cam pin with the cam grooves as the movable member moves in the axial direction, and the cam pin is rotatably supported by the insertion hole.

### BACKGROUND OF THE INVENTION

The present invention relates a forceps tool for endoscope used for endoscopic inspection and endoscopic surgery.

Conventionally, a forceps tool used for endoscope has been known. The forceps tool is used to grasp or cut living tissues with a pair of forceps when endoscopic inspection and/or endoscopic surgery is performed.

An example of such a forceps tool is disclosed in Japanese Patent Provisional Publication No. HEI 08-38495. Such a forceps tool is configured such that a pair of grasping members (forceps members) are rotatably supported by a pin member which is fixed to a supporting arm so that the grasping members rotate about the pin member to be opened/closed. The grasping members are provided with link arms, and an elongated hole, which serves as a cam groove, is formed on each of the ling arm. The pin member provided to a joint member is inserted in the elongated hole of each of the grasping members. The joint member is configured to move in forward/backward direction along an axial direction in association of a user operation of an operation unit provided to a proximal end of the forceps. When the pin member moved along the elongate holes as the joint member moves forward/backward, the grasping members open/close in accordance with an engaging status of the pin members with the elongated holes.

In the conventional configuration as described above, if there are certain clearances among the joint member, link aims and support arm, the pin member may not be inserted perpendicularly in the elongated holes. In such a state, the pin members may not slide inside the elongated holes smoothly and the grasping movement of the forceps may not be performed smoothly.

Therefore, in view of the smooth operation of the forceps, it is preferable that substantially no clearances are provided among the joint member, link arms and support arm.

On the other hand, if there are no clearances, it is likely that members may interfere with each other and/or frictional forces may generated between contacted members. In such case, the grasping members may not move smoothly.

If each member is manufactured with high dimensional accuracy, such a problem may be suppressed. However, to manufacture the elements with high dimensional accuracy, yield rate is lowered and manufacturing cost raises.

### SUMMARY OF THE INVENTION

In consideration of the above problem, the present invention is advantageous in that an improved forceps tool having smoothly movable forceps is provided without requiring extraordinarily high dimensional accuracy.

According to an aspect of the invention, there is provided a forceps tool for an endoscope, which is provided with a tube member, a support member secured to a distal end of the tube member, a support pin provided to the support member, a pair of forceps rotatably supported by the support pin, the pair of forceps being rotated about the support pin, a cam groove being formed on each of the pair of forceps, a movable member configured to axially move inside the tube in an axial direction thereof, an insertion hole formed on the movable member, a cam pin inserted through the insertion hole, both ends of the cam pin engaging with the cam grooves, respectively. The pair of forceps rotates about the support pin in accordance with engagement of the cam pin with the cam grooves as the movable member moves in the axial direction, and the cam pin is rotatably supported by the insertion hole.

Optionally, each of both ends of the cam pin may be formed to have a round shape.

Further optionally, the surface of the cam pin may be finished to improve a characteristic of sliding movement with respect to the cam grooves.

According to aspects of the invention, the forceps tool may be provided with a restriction member configured to restrict an axial displacement of the cam pin along an axis thereof. Optionally, the restriction member may include a pair of arms having the support pin therebetween.

Still optionally, the pair of forceps may be configured to be rotatable with respect to the tube about an axis of the tube.

Further optionally, the forceps tool may be for a flexible endoscope and the tube is flexible.

### BRIEF DESCRIPTION OF THE ACCOMPANYING DRAWINGS

FIG. 1A is a side view of a forceps tool for a flexible endoscope according to an embodiment of the invention.

FIG. 1B is a cross-sectional side view of the forceps tool shown in FIG. 1A.

FIGS. 2A-2C show side views of a distal end portion of the forceps tool shown in FIG. 1A, FIG. 2A showing a state where a forceps cup is opened, FIG. 2B showing a state where the forceps cup is closed, and FIG. 2C showing a state where the forceps tool is rotated about its axis by 90 degrees.

FIGS. 3A and 3B show cross-sectional side views of the distal end portion of the forceps tool, FIG. 3A showing a state where the forceps cup is opened, and FIG. 3B showing a state where the forceps cup is closed.

FIGS. 4A and 4B show cross-sectional views of the forceps tool taken along IV-IV plane of FIG. 3B, FIG. 4A show a state where a cam pin is inserted in each cam groove perpendicularly, while FIG. 4B shows a state where the cam pin is inserted in each cam groove obliquely.

### DETAILED DESCRIPTION OF THE EMBODIMENTS

Hereinafter, a forceps tool 1 according to an embodiment of the invention will be described with reference to the accompanying drawings.

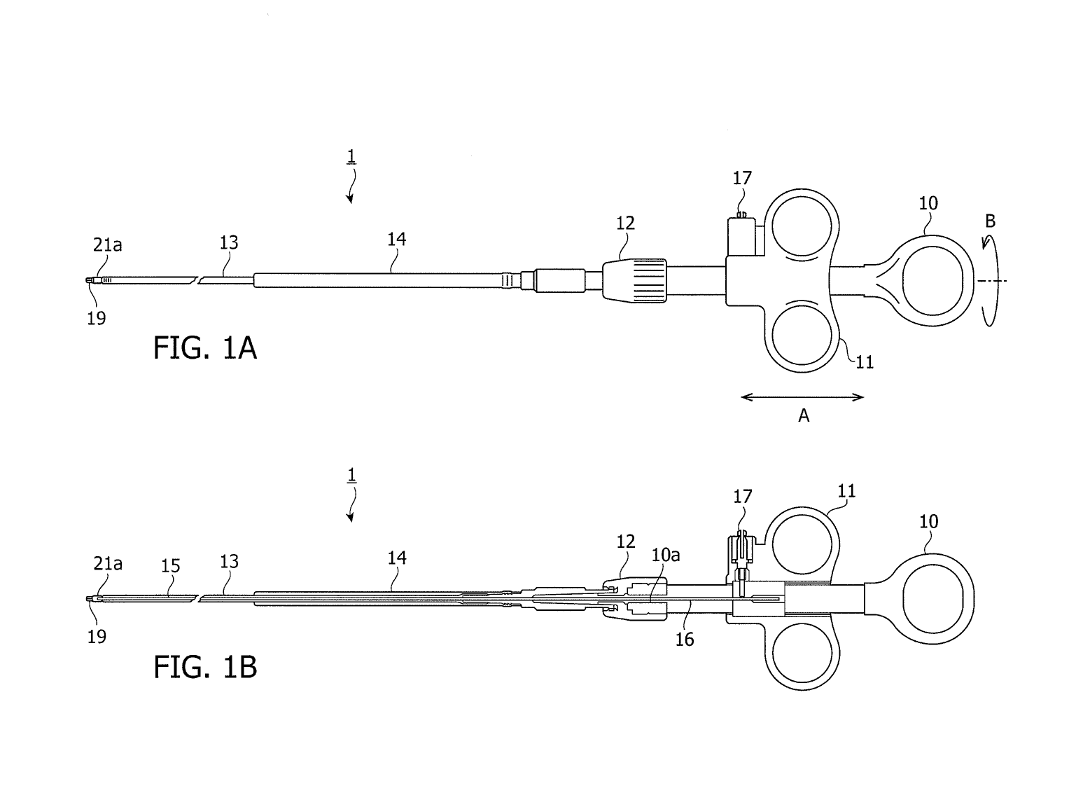

FIG. 1A is a side view of the forceps tool 1 for a flexible endoscope according to an embodiment of the invention. FIG. 1B is a cross-sectional side view of the forceps tool 1 shown in FIG. 1A. Generally, the forceps tool for the flexible endoscope has a smaller diameter than that for a rigid endoscope. For example, the outer diameter of the typical forceps tool for the flexible endoscope is 3 mm or less. Therefore, members of such a forceps tool 1 are generally small in size.

As shown in FIGS. 1A and 1B, the forceps tool 1 has an operation section 10. The operation section 10 slidably supports a sliding operation unit 11 so as to be slidable in a direction of arrow A. The operation section 10 rotatably supports a retaining ring 12, which is rotatable about an axis thereof. To the retaining ring 12, a proximal end of a flexible sheath 13 is fixedly connected. The flexible sheath 13 is an electrically insulating smoothly slidable flexible tube, which is made of, for example, ethylene tetrafluoride resin. The proximal end portion of the flexible sheath 13 is reinforced with a tube 14 surrounding the flexible sheath 13 so that the flexible sheath 13 is not bent easily.

Inside the flexible sheath 13, an electrically conductive operation wire 15, which is movable forward/backward in the axial direction, is inserted throughout the entire length of the flexible sheath 13. The proximal end portion of the operation wire 15 is coated with a reinforcing pipe 16, and is inserted in a slit 10a which is formed in the operation section 10 along its axis. The proximal end of the operation wire 15 is secured to the sliding member 11. Further, a high-frequency power source cord connection terminal 17 is provided to the sliding member 11. One end of the high-frequency poser source cord connection terminal 17 is connected to a high-frequency poser source (not shown), and the other end thereof is connected to the proximal end of the operation wire 15.

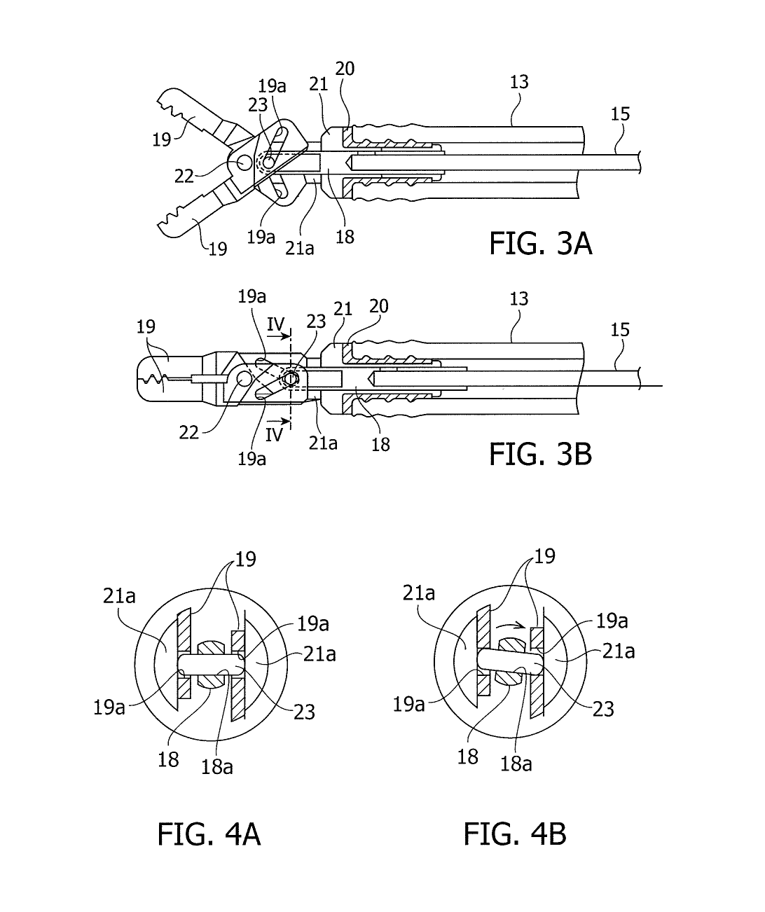

At the tip (distal end) of the operation wire 13, a joint member 18 is secured. The joint member 18 is coupled to the forceps cups 19 for collecting living tissues (see FIGS. 3A, 3B). When the user moves the sliding member 11 to slide in the direction of arrow A, with respect to the operation section 10, the operation wire 15 and the joint member 18 slide in the axial direction. In accordance with the forward/backward movement of the joint member 18, the forceps cups 19 are opened/closed.

According to the embodiment, the joint member 18 and the forceps cups 19 are made of stainless steel. The tip end (distal end) of the operation wire 15 is electrically connected to the forceps cups 19 via the joint member 18. When the high-frequency power source is driven, a high-frequency electrical current is applied to the forceps cups 19 via the high-frequency electrical cord connecting terminal 17, the operation wire 15 and the joint member 18.

When the user retains the retain ring 12 with his/her fingers and rotates the operation section 10 about its axis (i.e., in a direction of arrow B), the operation wire 15 rotates inside the flexible sheath 13, and the joint member 18 and the forceps cups 19 rotate integrally together with the operation wire 15. Thus, the user can change the direction of the forceps cups 19 arbitrarily by rotating the operation section 10. Because of the above structure, it is preferable that the operation wire 15 is made of torque wire which well transmits the rotational force.

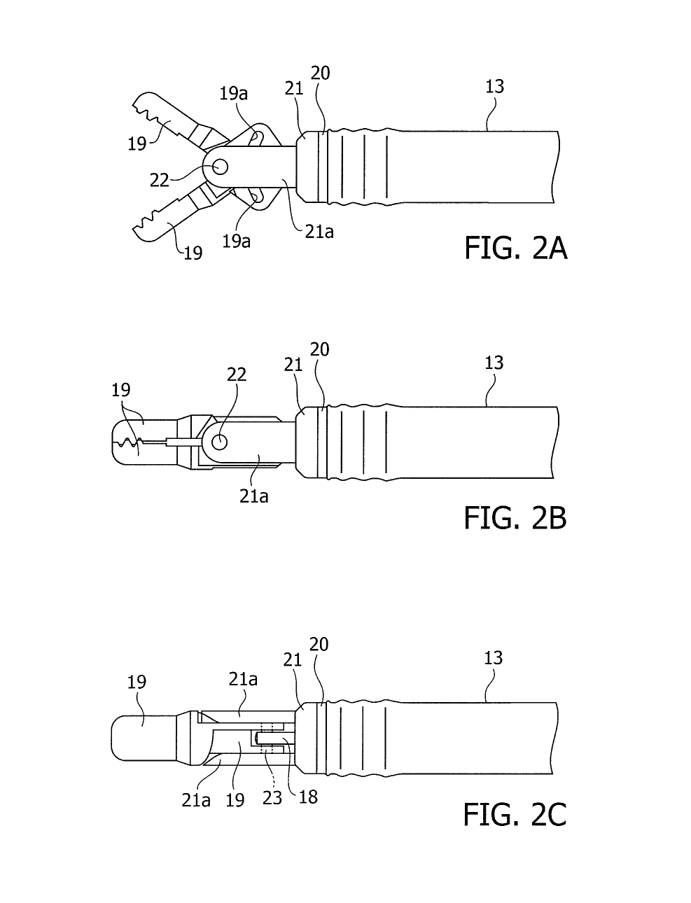

FIGS. 2A-2C show side views of a distal end portion of the forceps tool 1 shown in FIG. 1A. Specifically, FIG. 2A shows a state where a forceps cup is opened, FIG. 2B shows a state where the forceps cup 19 is closed, and FIG. 2C shows a state where the forceps tool 1 is rotated about its axis by 90 degrees.

FIGS. 3A and 3B show cross-sectional side views of the distal end portion of the forceps tool 1. FIG. 3A shows a state where the forceps cup 19 is opened, and FIG. 3B shows a state where the forceps cup 19 is closed.

At the distal end of the flexible sheath 13, a bracket 20 is secured. The bracket 20 is a hollow cylindrical member, and the joint member 18 and the end portion of the operation wire 15 are slidably supported inside the bracket 20. That is, the joint member 18 the end portion of the operation wire 15 can move in the axial direction inside the bracket 20. On the tip end of the bracket 20, a frame 21 is coupled such that the frame 21 is rotatable about the axis thereof. It should be noted that the bracket 20 and the frame 21 do not move in the axial direction when the operation section 11 is operated. The frame 21 is formed with a pair of supporting arms 21a. The pair of supporting arms 21a has through holes at the end portion thereof. A pin 22 is bridged between the pair of supporting arms 21a with its ends inserted through the through holes of the pair of supporting arms 21a. The ends of the pin 22 are deformed to have a larger diameter than that of the through holes so that the pin 22 supported between the pair of supporting arms 21a. The forceps cup 19 is arranged between the pair of supporting arms 21a, and rotatably supported by the pin 22.

FIGS. 4A and 4B show cross-sectional views of the forceps tool 1 taken along IV-IV plane of FIG. 3B. As shown in the drawings, the joint member 18 is formed with a through hole 18a in which a cam pin 23 is inserted. The forceps cup 19 is formed with cam grooves 19a which are elongated openings, in which the ends of the cam pin 23 are engageably inserted.

When the user operates the sliding member 11 to slide, with respect to the operation section 10, in the direction of arrow A (see FIG. 1), the cam pin 23 moves forward/backward together with the joint member 18 in the axial direction. Since the both ends of the cam pin 23 are inserted in the cam grooves 19a, the forceps cup 19 rotates about the pin 22 (i.e., the forceps cup is opened/closed) due to the engagement of the cam pin 23 with the cam grooves 19a.

Generally, the components of the forceps for flexible endoscope are smaller than those for rigid endoscope, and malfunctions may likely occur due to interference, contact and/or friction among the components. Therefore, as shown in FIG. 4A, certain clearances are provided among components (e.g., the joint 18, the forceps cup 19 and the supporting arms 21a). However, because of such clearances, there is a possibility that the cam pin 23 engages with the cam grooves 19a in a state that the cam pin 23 is not inserted perpendicularly to the cam grooves 19a. In such a case, that cam pin 23 may stick with respect to the cam grooves 19a, or the cam mechanism (i.e., the engagement of the cam pin 23 with the cam grooves 19a) may malfunction.

According to the embodiment, to avoid the above problem (e.g., the stuck condition), the cam pin 23 is merely inserted through the insertion hole 18a and is not fixed therein. That is, the cam pin 23 is rotatable with respect to the joint member 18 about the axis thereof. With such a configuration, even if the cam pin 23 is inclined and slidable contact with the cam grooves 19a does not function well as shown in FIG. 4B, the cam pin 23 itself rotates and relative movement of the cam pin 23 and the cam grooves 19a is performed smoothly. Therefore, open/close movement of the forceps cup 19 is performed smoothly. It should be noted that the axial movement of the cam pin 23 when operated to move forward/backward in the direction of arrow A is restricted by the supporting arms 21a. Therefore, the cam pin 23 does not drop out from the insertion hole 18a.

In order to reduce suppress the stuck condition above, the end portions of the cam pin 23 are rounded (e.g., formed to have a hemispherical shape). Further, in order to improve sliding performance of the cam pin 23 with respect to the insertion hole 18a and/or cam grooves 19a, the surface of the cam pin 23 may be finished to improve slidability, or a characteristic of sliding movement with respect to the cam grooves 19a. For example, the cam pin 23 may be formed with a DLC (Diamond-Like-Carbon) coat.

According to the above configuration, since the components of the above-described cam structure (i.e., the joint member 18, the cam grooves 19a and the cam pin 23) each has a simple structure, manufacturing cost can be suppressed. Further, the cam pin 23 is not secured to the joint member 18, which also suppresses the manufacturing cost since no additional process of securing the cam pin 23 to the joint member 18 is required.

It should be note that the above-described embodiment is merely an exemplary embodiment, and various modifications may be realized without departing from the scope of the invention.

This application claims priority of Japanese Patent Application No. P2010-186991, filed on Aug. 24, 2010. The entire subject matter of the applications is incorporated herein by reference.

What is claimed is:

1. A forceps tool for an endoscope, comprising:
a tube member;
a support member secured to a distal end of the tube member;
a support pin provided to the support member;
a pair of forceps rotatably supported by the support pin, the pair of forceps being rotated about the support pin, a cam groove being formed on each of the pair of forceps;
a movable member configured to axially move inside the tube in an axial direction thereof;
an insertion hole formed on the movable member;
a cam pin inserted through the insertion hole, both ends of the cam pin engaging with the cam grooves, respectively,
wherein the pair of forceps rotates about the support pin in accordance with engagement of the cam pin with the cam grooves as the movable member moves in the axial direction, and
wherein the cam pin is rotatably supported by the insertion hole.

2. The forceps tool according to claim 1, wherein each of both ends of the cam pin is formed to have a round shape.

3. The forceps tool according to claim 1, wherein the surface of the cam pin is finished to improve a characteristic of sliding movement with respect to the cam grooves.

4. The forceps tool according to claim 1, which is provided with a restriction member configured to restrict an axial displacement of the cam pin along an axis thereof.

5. The forceps tool according to claim 4, wherein the restriction member includes a pair of arms having the support pin therebetween.

6. The forceps tool according to claim 1, wherein the pair of forceps are configured to be rotatable with respect to the tube about an axis of the tube.

7. The forceps tool according to claim 1, wherein the forceps tool is for a flexible endoscope and the tube is flexible.
